(19) United States
(12) Patent Application Publication
(10) Pub. No.: US 2016/0022872 A1
(43) Pub. Date: Jan. 28, 2016

(54) CELL-BASED COMPOSITIONS, CELL-BASED BANDAGE DEVICES AND SYSTEMS AND METHODS OF TREATMENT THEREWITH

(71) Applicant: UNIVERSITY OF PITTSBURGH-OF THE COMMONWEALTH SYSTEM OF HIGHER EDUCATION, Pittsburgh, PA (US)

(72) Inventors: ALAN WELLS, Pittsburgh, PA (US); CECELIA CHRISTINA YATES-BINDER, Pittsburgh, PA (US); DONALD P. TAYLOR, Pittsburgh, PA (US)

(21) Appl. No.: 14/772,501

(22) PCT Filed: Mar. 5, 2014
(86) PCT No.: PCT/US2014/020631

§ 371 Date: Sep. 3, 2015

Related U.S. Application Data
(60) Provisional application No. 61/773,650, filed on Mar. 6, 2013.

(51) Int. Cl. A61L 27/38; A61L 27/18; A61L 27/58; A61L 27/54; C12N 11/04; C12N 11/08

(52) U.S. Cl. CPC A61L 27/3834; C12N 11/04; C12N 11/08; A61L 27/58; A61L 27/54; A61L 27/18; A61L 2300/412

(57) ABSTRACT

A composition includes a biodegradable matrix, cells within the biodegradable matrix and at least one bioactive agent having limited mobility relative to the cells within the biodegradable matrix. The at least one bioactive agent is bioactive within the biodegradable matrix for regulation of the cells to enhance cell survival.

### CROSS-REFERENCE TO RELATED APPLICATIONS

This application claims benefit of U.S. Provisional Patent Application Ser. No. 61/773,650, filed Mar. 6, 2013, the disclosure of which is incorporated herein by reference.

### GOVERNMENTAL INTEREST

This invention was made with government support under grant no. GM63569 awarded by the National Institute of General Medical Sciences. The government has certain rights in this invention

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof The disclosure of all references cited herein are incorporated by reference.

Although wound physiology is well understood, only modest product improvements for wound treatment have emerged. Even so, the relatively new market of Advanced Wound Care is currently $US 1.3B, and is projected to grow to $US 2.1B by 2017 (of the $US5B total wound care market). A number of products in this market attempt to modulate the wound healing process by incorporating specialized biomaterials, cell therapies, or antimicrobial agents to accelerate wound healing. Unfortunately, advanced wound products fail to overcome the damaging inflammatory phases plaguing wounds that cannot heal properly. Persisting wound inflammation inhibits wound bed remodeling, recruitment of vasculature and growth factor availability.

### SUMMARY

In one aspect, a composition including a biodegradable matrix, cells within the biodegradable matrix and at least one bioactive agent having limited mobility relative to the cells within the biodegradable matrix. The at least one bioactive agent is bioactive within the biodegradable matrix for regulation of the cells (for example, via interacting with or signaling cell receptors) to enhance cell survival. The at least one bioactive agent may, for example, be a matrikine or a matrikine fragment which is immobilized upon the cells or is immobilized upon or within the biodegradable matrix.

In a number of embodiments, the cells are cells which can differentiate into at least one other cell type. The cells may be multipotent. The cells may, for example, include at least one of mesenchymal stem cells, tissue stromal cells, tissue epithelial cells (including, but not limited to, keratinocytes, hair follicle cells, hepatocytes, and other tissue parenchymal cells) or endothelial progenitor cells.

In a number of embodiments, the matrikine or the matrikine fragment either (i) do not induce differentiation or (ii) inhibit differentiation while the cells are within the biodegradable matrix. In a number of embodiments, the composition may further include cells or another agent that facilitates or directs differentiation.

The matrikine or the matrikine fragment (or other bioactive agent) may, for example, be tethered to the biodegradable matrix. In a number of embodiment the matrikine or the matrikine fragment (or other bioactive agent) is covalently bonded to the biodegradable matrix. In a number of embodiments, the matrikine or the matrikine fragment (or other bioactive agent) is non-covalently bonded to the biodegradable matrix. The matrikine or matrikine fragment (or other bioactive agent) may be adhered to the cells.

The matrikine or the matrikine fragment may, for example, selected from the group of a tenascin, a laminin, a fibronectin, a thrombospondin and an elastin, or fragments thereof. In a number of embodiments, the matrikine or the matrikine fragment is selected from the group of tenascin-C and laminin beta 1. The matrikine or the matrikine fragment may, for example, include EGF-like repeat regions or domains. The matrikine of the matrikine fragment may, for example, include EGF-like repeat regions of a tenascin or EFG-like repeat regions of a laminin. In a number of embodiments, the matrikine or the matrikine fragment includes EGF-like repeat regions of tenascin-C or EFG-like repeat regions of a laminin beta 1.

In a number of embodiments, the biodegradable matrix includes a biodegradable polymer matrix. The biodegradable polymer matrix may, for example, be formed via polymerization a polymerizing reactant that is adapted to undergo a free radical polymerization. In a number of embodiments, the at least one bioactive agent is covalently attached to a polymerizing molecule in forming the polymerizing reactant. The polymerizing molecule retaining the ability to undergo free radical polymerization after attachment of the at least one bioactive agent thereto. In a number of embodiments, at least one interacting agent that is adapted to interact with the at least one bioactive agent (to limit the mobility thereof relative to the cells) is covalently attached to a polymerizing molecule in forming the polymerizing reactant. The polymerizing molecule retaining the ability to undergo free radical polymerization after attachment of the at least one interactive agent thereto.

The polymerizing reactant may, for example, include a residue of (or the, polymerizing molecule may, for example, be) dihydroxyphenyl-L-alanine (DOPA), a derivative of dihydroxyphenyl-L-alanine, histidine, a derivative of histidine, lysine, a derivative of lysine, tryptophan, a derivative of tryptophan, tyrosine or a derivative of tyorsine. In a number of embodiments, the polymerizing reactant may, for example, include a residue of (or the, polymerizing molecule may, for example, be) dihydroxyphenyl-L-alanine (DOPA).

The biodegradable polymer matrix may, for example, include a polymer of polyethylene glycol and dihydroxyphenyl-L-alanine (DOPA) or a derivative of dihydroxyphenyl-L-alanine. In a number of embodiments, the biodegradable polymer matrix includes a polymer of polylactic acid.

In another aspect, a method of forming a composition includes incorporating at least one bioactive agent within a biodegradable matrix, and incorporating cells within the biodegradable matrix, wherein the at least one bioactive agent can come into contact with the cells and has limited mobility relative to the cells, and wherein the at least one bioactive agent is bioactive within the biodegradable matrix for regulation of the cells to enhance cell survival.

In a further aspect, a method of treatment includes application of a composition as described above to living tissue.

In still a further aspect, a tissue application system includes a composition as described above. The tissue application system may, for example, further include a cover adapted to be placed over the composition upon application of the composition to tissue.

The present devices, systems, methods and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a prepolymer of the present invention synthesized from a di-functional polyethylene glycol core molecule, a diisocyanate linker molecule and L-3,4-dihydroxyphenylalanine (L-DOPA or DOPA).

FIG. 1B illustrates cross-linking pathways for the DOPA-containing biodegradable polymers hereof.

FIG. 1C further illustrates cross-linking pathways for the DOPA-containing compounds of the present invention.

FIG. 1D illustrates a polymer formed using hexamethylene diisocyanate (HDI), PEG and DOPA, wherein the DOPA is activated and conjugated with a matrikine.

FIG. 2 illustrates the results of degradation studies of a prepolymer, a PEG-polymer and a PEG-Polymer plus Tenascin (TNC) hereof.

FIG. 3 illustrates photographs of a biodegradable polymer hereof embedded within an induced mouse wound at three times (0 minutes, 20 minutes and 40 minutes).

FIG. 4A illustrates flow cytometric quantification of phycoerythrin labeled MSC with triple-stained cells in an anoxic gel implant in mice, demonstrating that the MSC survive for extended times when supported by tethered EGF.

FIG. 4B illustrates flow cytometric quantification of FITC/HLA+cells used to detect MSC survival in wound bed at 3 days, 7 days and 21 days and shows that the MSC survive more and longer with tenascin C (inclusive of EGF-like repeats).

FIG. 5 illustrates the results of studies of wound maturation at 7 days, 21 days and 60 days, which demonstrate that PEG-polymer+TNC/MSC improved outcomes.

FIG. 6A illustrates the results of studies of dermal maturation at 7 days, 21 days and 60 days.

FIG. 6B illustrates the results of studies of normalized percent collagen content at 7 days, 21 days and 60 days.

FIG. 7A illustrates flow cytometric analysis of CD3+ expressing cells in the wound bed of wounds treated with the PEG-Polymer+TNC/MSC as compared to untreated wounds, which demonstrates that PEG-polymer+TNC/MSC limited nonspecific inflammation that is detrimental to wound healing, which is also shown FIG. 7B.

FIG. 7B illustrates flow cytometric analysis of CD4+Foxp3+ expressing cells in the wound bed of wounds treated with the PEG-Polymer+TNC/MSC as compared to untreated wounds.

FIG. 8 illustrates flow cytometric analysis of cells expressing CD31+ in the wound bed of wounds treated with PEG-Polymer+TNC/MSC as compared to untreated wounds, which demonstrates that PEG-polymer+TNC/MSC improves the blood vessel content of the wound.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely representative of embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a matrikine” includes a plurality of such matrikines and equivalents thereof known to those skilled in the art, and so forth, and reference to “ the matrikine” is a reference to one or more such matrikines and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

In a number of representative embodiments, cell-based polymeric wound/tissue gels hereof provide cell delivery to effect cellular therapies for treating, for example, chronic or traumatic wounds. In that regard, devices, systems, compositions and/or methods hereof may, for example, be used to intervene during early and chronic wound inflammatory responses that otherwise contribute to delayed wound healing, anti-angiogenesis, aberrant stromal cell recruitment, infection, or scarring. The devices, systems, compositions and/or methods hereof limit or overcome a number of limitations associated with the inflammatory response associated with wounded or damaged tissue, thereby healing wounds faster, with significant reductions in patient morbidity, mortality and healthcare costs. Although representative embodiments of devices, systems, compositions and/or methods hereof are discussed herein in connection with wound treatment, the devices, systems, compositions and/or methods hereof are well suited for delivery of cells to tissue with the delivery both for the cells to provide directly and indirectly via secreted factors in generally any application in which cells are to be delivered.

Composition hereof include a biodegradable matrix incorporating or including cells and at least one bioactive (or biologically active) agent having limited mobility relative to the cells. The mobility of the bioactive agent relative to the cells may, for example, be limited by interacting/attaching the biologically active agent with/to at least one of the biodegradable matrix or the cells. Although individual biologically active agents hereof may bind to cells with low affinity, limiting the mobility of the bioactive agents relative to the cells provides for high avidity (that is, the accumulated strength of multiple affinities of individual non-covalent binding interactions between the bioactive agent and the cells). The bioactive agent is biologically active within the biodegradable matrix for regulation of the cells to enhance cell survival. The cells may, for example, have the ability to differentiate into at least one other cell type. The cells may, for example, be multipotent cells (that is, cells that can differentiate into a number of different types of cells). However, in a number of embodiments, the cells need not differentiate to provide beneficial effect. The at least one bioactive agent may, for example be a matrikine or active fragment thereof, whether natural or artificially-generated.

As used herein, the term matrikine refers to an extracellular matrix-originating or matricellular peptide with bioactivity to regulate cell activity. Matrikines include signaling domains that activate cell receptors. The term matrikine includes “natural” matrikines, which signal directly from the extracellular milieu and “cryptic” matrikines (sometimes called matricryptins) that require (partial) proteolytic processing or unwinding to reveal the ligand or to release the ligand from its extracellular matrix parent protein. Processes that can lead to the exposure of cryptic cleavage sites and the subsequent generation of matrikines include enzymatic degradation, mechanical forces, adsorption and heterotypic binding to other molecules, multimerization and self-assembly, and induced conformational changes. Matrikines include, for example, those of a tenascin, a laminin, a fibronectin, a thrombospondin or an elastin. In a number of representative embodiments set forth herein, the cells are mesenchymal stem cells, tissue stromal cells or endothelial progenitor cells and the bioactive agent is the matrikine tenascin C (TNC), or one or more matrikine fragment(s) with EGF-like repeats.

Tenascin-C or TNC may reduce the number of focal adhesions and decrease cell adhesiveness in several cell types. To test for MSC adhesiveness on TNC, an inverted centrifugation assay was performed 2 hours post-seeding MSC on tissue culture plastic and the number of cells attached after centrifugation was compared to number of cells attached on a similarly coated surface that had not undergone centrifugation. MSCs grown on TNC were found to detach from surfaces after inverted centrifugation. To increase adhesiveness of cells on TNC we coated mixed Collagen I, a matrix naturally occurring in the in vivo bone environment and one that promotes MSC attachment and spreading, in a 1:1 ratio with TNC for coating substrates. Cells on TNC were only half as adhesive as cells on Collagen I. Growing cells on a mixed matrix including Collagen I and TNC significantly increased MSC adhesiveness. In a number of studies hereof, a combination of Collagen I and TNC was used to increase TNC adherence to cells.

During wound healing, endogenous MSC proliferation and differentiation are temporally distinct events. Use of an exogenous survival factor, which promotes (or at the least allows for) proliferation, but does not induce differentiation would cause MSC expansion post-incorporation into tissue, promoting sufficient numbers of cells to form to regenerate the wounded tissue, whether this is via the transplanted cells or endogenous cells as a result of signals from the transplanted cells. We tested if the extracellular matrix or ECM factors had any influence on MSC proliferation. TNC combined with Collagen I, like Collagen I by itself, was seen to promote both imhMSC and prhMSC proliferation 96 hours post-seeding, relative to cell numbers initially plated.

We then studied whether TNC by itself, in the absence of specific differentiation cues, promotes MSC differentiation. We examined osteoblast or adipose cell formation from MSC grown on TNC and Collagen (Col I), in the presence of either proliferation or differentiation media. Von Kossa staining of MSC grown for 30 days in proliferation media showed that neither Collagen I by itself nor TNC and Collagen I caused hydroxyapatite deposition. In the presence of differentiation media, however, TNC did not interfere with osteogenic differentiation. Similarly TNC did not promote adipocyte formation on its own. Further, TNC did not prevent adipocyte formation in the presence of adipogenic inducers. The matrix mix of TNC and Collagen I thus supported MSC attachment and growth and did not cause MSC to differentiate. The matrix mix of TNC and Collagen I also did it interfere with differentiation.

MSCs are most susceptible to cell death via the Fas death pathway. In a number of studies, we induced cell death in imhMSC and prhMSC by treating with FasL and found intense capsase3 activation as highlighted by FLICA® assay (Fluorescent Labeled Inhibitor of Caspases; Immunochemistry Technologies, LLC of Bloomington, Minn.) after 8 hours (for imhMSC) or 12 hours (for prhMSC) of treatment. Cells were also treated with a low concentration of the protein synthesis inhibitor cycloheximide (CHX) in addition to FasL. Cycloheximide, being a protein synthesis inhibitor, increases cell stress, imitating the starvation stress seen in vivo in a wound setting. Cells grown on tissue culture plastic with no matrix displayed caspase-3 activation after treatment with FasL or CHX and FasL. Cells grown on Collagen I and TNC displayed a survival advantage and substantially reduced cell death in the presence of FasL or CHX and FasL. TNC (1 Lg/cm2) was found to be optimal to promote survival in MSC in the presence of FasL based on a dose response, as determined by TUNEL and FLICA staining. We attributed the protective effect to TNC in the matrix since MSC grown on Collagen I surfaces did not show protection to MSC in the presence of FasL or CHX and FasL. Similarly we observed that TNC and Collagen I, but not Collagen I by itself, protected MSC from nuclear DNA damage induced by FasL, as indicated by terminal deoxynucleotidyl transferase dUTP nick end labeling or TUNEL assay. These results indicated that TNC plays a role in increasing cell survival in MSC in the presence of threats like FasL.

Since TNC contains both epidermal growth factor like or EGF-like (EFGL) repeats, which can bind and activate epidermal growth factor receptor or EGFR as well as fibronectin like (FNL) repeats which can bind and signal integrins, we studied which moieties provided the survival effects. An EGF-like domain is an evolutionary conserved protein domain, which derives its name from the epidermal growth factor where it was first described. The EGFL comprises about 30 to 40 amino-acid residues and has been found in a large number of mostly animal proteins. The EGF-like domain includes 6 cysteine residues which in the epidermal growth factor have been shown to form 3 disulfide bonds.

After preparation of coated surfaces and prior to seeding of MSC, neutralizing antibodies to the EGFL and FNL domains were added for 24 hours Immortalized hMSC were then seeded for 24 hours, kept treated or untreated with FasL for 8 hours, followed by a FLICA assay to test for caspase3 activation. Presence of neutralizing antibodies for FNL and EGFL was confirmed prior to seeding of cells. Blocking of FNL allowed for a small amount of caspase3 activation but much less than that without antibody blockade. On the other hand, the neutralization of EGFL permitted cell death to the extent of no blockade. MSCs seeded on Collagen I alone with no neutralizing antibody and treated with FasL showed caspase 3 activation as shown earlier. Addition of neutralizing antibodies did not have a toxic effect on MSCs. This result was confirmed by growing MSCs on Collagen I and TNC coated surfaces with neutralizing antibody, untreated, and testing for caspase3 activation. These results indicate that although both EGFL and FNL repeats of TNC are involved in survival signaling in MSC, the dominant survival effect is via EGFL.

We next studied whether TNC contributes to survival of MSCs by sequestering FasL and prevention of FasL from binding its receptor Fas on MSC. We grew MSCs inside two cloning cylinders placed in wells of a 6-well dish, enclosing areas without any ECM, or with Collagen I and TNC, on which imhMSC were grown. One well had Collagen I and TNC coating surrounding the cloning cylinders, covered with media and FasL. The second well had only media with FasL surrounding the cloning cylinders, with no TNC. When the cloning cylinders were taken out and media came in contact with cells within the cloning cylinders, there was comparable cell death seen in cells grown on uncoated surfaces in both wells. Cells coated on TNC did not show caspase3 activation as seen earlier and expected. This indicated that TNC was neither sequestering nor neutralizing FasL.

To determine that EGFR is the operative receptor for the survival advantage, prhMSC were grown on Collagen I and TNC coated surfaces, and treated with inhibitors of EGFR activation PD153035 (an inhibitor of the EGF receptor tyrosine kinase), Erk activation PD98059 (a selective, cell-permeable inhibitor of MAP kinase (MEK)), or Akt activation LY294002 (an inhibitor of phosphoinositide 3-kinases), 30 minutes prior to addition of FasL. The inhibitors were present during the entire 8 hours of FasL treatment, after which a FLICA assay was performed. Blocking of either EGFR or its downstream pathways of Akt and Erk blocked the survival advantage that TNC and Collagen I provide MSCs in the presence of FasL. The controls of only inhibitor treatment PD153035, or PD98059 or LY294002 on MSC grown on Collagen I and TNC, did not have a toxic effect on cells. As seen earlier MSC grown on Collagen I and TNC and treated with FasL, or CHX and FasL survived. These results indicate that activation of EGFR and its downstream pathways of Erk and Akt by TNC are necessary for promoting survival in MSC.

Upon determining that TNC provided a survival effect on MSC in the presence of FasL, and that this mapped to the EGFL, we studied if Tenascin C was able to activate EGFR on the surface of MSC and if this activation was sustained. MSCs have low levels of EGFR, which may limit activation by the ultra-low affinity EGFL. In a number of studies, immortalized hMSC were plated on surfaces with Collagen I alone or Collagen I and TNC for 24 hours. After 24 hours, (considered 0 minute), immunofluorescence for phospho-EGFR (red fluorescence) and the early endosome marker EEA1 (green fluorescence) was done on these samples. Immortalized hMSC on Collagen I and TNC showed phospho-EGFR, while cells on Collagen I alone did not show phospho-EGFR staining showing specific activation of EGFR by TNC. Immortalized hMSC seeded on Collagen I were treated with EGF, the prototypal EGFR ligand, known to cause EGFR internalization for 15, 30 and 60 minutes prior to fixation, to induce phosphorylation of EGFR. At the same time points, immunofluorescence was done on cells grown on Collagen I and TNC. As expected, cells treated with EGF stained positive for phospho-EGFR after 15 minutes of treatment, and phospho- EGFR co-localized with the endosome marker EEA1 at 15 and 30 minutes of treatment, indicating activation and internalization of EGFR by EGF. At 60 minutes post-treatment with EGF, there was very little phospho-EGFR signal indicating near complete internalization of receptors. phospho-EGFR on TNC and Collagen I surfaces however continued to be expressed at 15, 30 and 60 minutes, indicating sustained activation of EGFR on the cell surface by TNC compared to EGF.

Since TNC was found to cause sustained EGFR signaling in MSC, we tested if this signal translated to sustained downstream signals in MSC related to survival. We examined that of Erk Immortalized hMSC were plated on surfaces with Collagen I alone or Collagen I and TNC for 24 hours. After 24 hours, (considered 0 minute), immunofluorescence for phospho-Erk (green fluorescence) was tested either with or without the EGFR inhibitor PD153035. TNC was found to activate phosho-Erk in MSC at 0, 30 and 60 minutes. In the presence of EGFR inhibitor, this activation was reduced but not completely inhibited, indicating the presence of other modes of Erk activation in MSC. There was Erk activation seen in the presence of Collagen I alone, however significantly less than that with TNC. These results suggest that TNC causes sustained EGFR and Erk signaling, promoting survival in MSC.

To assess the survival advantage TNC provides to MSC during cell transplantation, in several studies, a hydrogel delivery system to deliver carboxycyanine-tracked human MSC in a mouse wound model. Full thickness 8 mm punch biopsy wounds were created in wild-type mice. These wounds were then treated with hyaluronic acid (HA) alone, human MSC in HA, human MSC in HA-Col, or human MSC in HA-Col-TNC. At days 3, 7, 14, and 28 wound biopsies were assessed for the survival and maintenance of carboxycyanine-tracked human MSC. Donor cells delivered in the HA-Col-TNC−MSC gel exhibited strong survival within the wound bed at day 7 post-wounding. The cryosections did not display carboxycyanine positive MSCs in HA without Tenascin-C. This trend was seen on Day 14 as well as Day 28. These data further support the in vitro finding that suggest TNC promotes survival of MSC and provides a strategy for maintaining MSC survival in wounds.

Immunofluorescent sections of the wound tissue demonstrated increased MSC survival by TNC, but are not quantitative. The survival advantage of TNC on MSC in the wound was thus further investigated by flow cytometry analysis of single cells isolated from wound tissue. Wound tissue was treated with liberase to separate out single cells, and the carbocyanine tracked MSCs were analyzed by Flow Cytometry using the PE channel. Flow cytometry of dissociated wound beds at days 3, 7, 14 and 28 post-wounding validated the histological analyses demonstrating higher survival of HA-Col-TNC−MSC gel delivery versus that of HA-MSC or HA-Col-MSC by a significantly higher percentage. At Day 7, there were 21% PE positive cells in the HA-Col-TNC−MSC population vs 10% positive cells in HA-Col-MSC and 8% positive cells in the HA-MSC population. Taken together, Tenascin-C and Collagen I extended a survival effect to MSC's not just in vitro, but also in vivo in a wound site.

The above cell-based polymeric wound gel studies including matrikines or matrikine fragments and mesenchymal stem cells (MSC) that therapeutically cooperate to treat chronic or traumatic wounds were augmented with studies of the matrikine TNC and MSC in another polymer gel that forms a tissue-like rheology more suitable for wound bed topical application (for example, with degradable properties over a clinically relevant therapeutic window). Methods for synthesizing polymer matrices/gels suitable for use herein are, for example, described in U.S. Pat. No. 8,029,774 and PCT International Patent Application Publication No. WO/2007/005792, the disclosure of which are incorporated herein by reference. Such polymers may, for example, exhibit several or more of the following desirable characteristics: the material is liquid prior to use or polymerization; solidifies in a controllable time frame when applied to wound; is nonirritating locally; is nontoxic systematically in the amount required to achieve an effective wound closure; exhibits hemostatic properties; alleviates pain; has appropriate flexibility (elastomeric); and/or is biodegradable so that the material does not disturb wound healing. As used herein, the term “biodegradable” refers generally to the ability of the material to be broken down (especially into innocuous degradation products) over time in the environment of use (that is, within the body/wound).

The terms polymeric network, matrix and gel are used interchangeably herein and refer to a polymer system (for example, a crosslinked polymeric network). In a number of embodiments, polymers gels hereof are produced in a two part synthetic scheme with gelation preferably being catalyzed by ionic silver (Ag) containing catalyst. Silver, which may distribute through the polymer matrix passively, is widely used as an antimicrobial.

Crosslinked polymer networks, matrices or gels hereof may, for example, be formed with a “synthetic component” comprising a prepolymer component formed from one or more multi-functional core molecules. Functional groups of the core molecule(s) are reacted with a first functional group of one or more multifunctional linker or spacer molecules. A second functional group of the linker molecule is reacted with a functional group of one or more molecules that can undergo a polymerization (for example, a free radical polymerization) under relatively benign condition. Preferably, the linker molecule is chosen so that biodegradable covalent bonds are formed between the linker molecule and the core molecule and between the linker molecule and the polymerizing molecule. In a number of embodiments, the degradation products (generally, the core molecule or a derivative thereof, the linker molecule or a derivative thereof and the polymerizing molecule or a derivative thereof) are biocompatible.

The polymerizing molecule retains the ability to polymerize after reaction with the linker molecule. The polymerizing molecules may, for example, include one or more functional groups suitable to undergo a free radical polymerization as described herein (for example, a hydroxyphenyl group as present in DOPA and tyrosine). The polymerizing molecules may also include one or more functional groups suitable to effect covalent attachment (either directly or indirectly through one or more chemical moieties) of, for example, a linker molecule thereto or covalent attachment of a biologically active agent (for example, a matrikine) and/or another agent thereto.

In several embodiments of the present invention, one or more multi-isocyanate functional molecules (that is, molecules including multiple isocyanate functional groups) were used as the linker molecule(s). Aliphatic multi-isocyanate functional molecules may, for example, be used. Examples of suitable aliphatic multi-isocyanates include, but are not limited to, hexamethylene diisocyanate (HDI) and butane diisocyanate. Multi-isocyanate functional molecules may, for example, derived from one or more biocompatible multi-amine functional precursors. In several embodiments, the multi-amine functional precursor compounds of the multi-isocyanate functional compounds are biocompatible amino acids or biocompatible derivatives of an amino acids. For example, the multi-amine functional precursor compound can be lysine. The multi-isocyanate functional compounds can, for example, include lysine di-isocyanate or lysine di-isocyanate derivatives (for example, an alkyl ester of lysine-diisocyanate such as the methyl ester or the ethyl ester). The multi-isocyanate functional compounds can also include lysine tri-isocyanate or a derivative of lysine tri-isocyanate. Certain dipeptides (two amino acids linked by an amide linkage) can also be used. For example, lysine can be combined in a dipeptide with another amino acid (for example, lysine-valine, lysine glysine etc.). Another example of a multi-amine functional compound suitable for use in the present invention is putrescine (diamino butane).

In the case that one or more multi-isocyanate compounds are used as the linker molecule(s), the core molecule(s) include reactive hydrogen functional groups (for example, hydroxy groups, primary amine groups, secondary amine groups or thiol groups). Examples of suitable core molecules include, but are not limited to, glycerol, diglycerol, ascorbic acid, a saccharide (for example. glucose, lactose etc.), pentaerythritol, xylitol, arabitol, fucitol, ribitol, gluconic acid, glucosamine, sorbitol, mannitol, sugar alcohols generally, a steroid and biocompatible multi-functional polymers (for example, multi-hydroxy functional polyethylene glycol or PEG, a polyamino acid, a polyether or a polyester). The core molecule, the linker molecule and the polymerizing molecule may, for example, be selected so that the synthetic components is liquid or flowable prior to curing/polymerization.

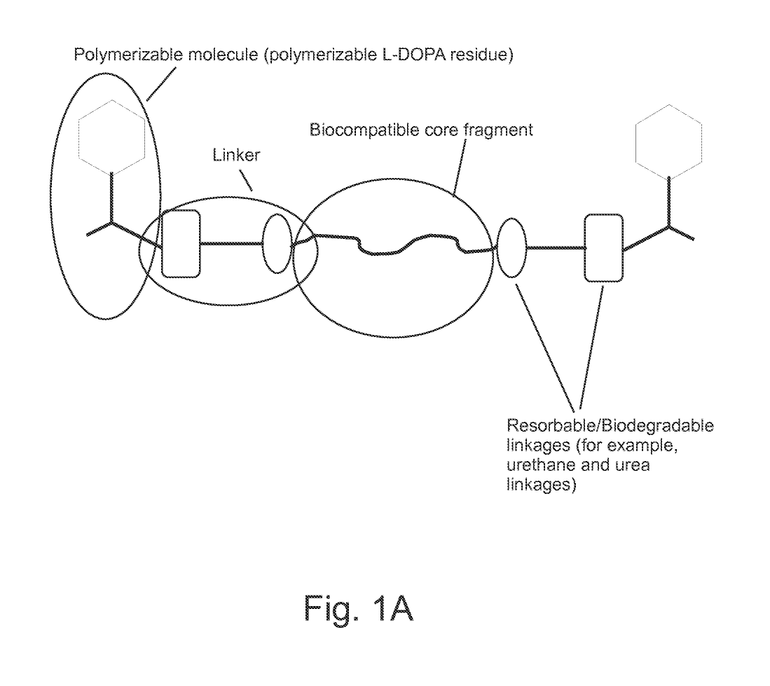

The functionality of the core material determines the functionality of the resulting network after curing as illustrated, for example, in FIG. 1A, which illustrates a DOPA-containing prepolymer formed from a di-functional core molecule (for example, di-functional PEG). In a number of embodiments, the core molecule is reacted with at least a stoichiometric amount (NCO:OH=2:1), and typically an excess of a diisocyanate (for example, HDI or LDI), capping the reactive hydrogen groups, creating in the process a resorbable/degradable urethane link, and terminating the arms of the core with a reactive isocyanate group. The core conjugate is reacted with, for example, a polymerizable molecule such as L-DOPA, creating a multi-functional, DOPA-terminated precursor. Dialysis may, for example, be used to purify the product. In the case of the use of HDI as the diisocyanate, each of the synthetic component precursors has the general formula Core-(HDI-DOPA)ₓ, wherein x is the functionality of the core molecule. In the case of the use of di-functional PEG as the core, the precursors have the general formula PEG-(HDI-DOPA)₂. Other biocompatible compounds such as amino acids (including, but not limited to, histidine, lysine, tryptophan, and tyrosine) that can form crosslinks via free-radical chemistry, may be substituted for or potentially used in connection with L-DOPA.

An efficient redox initiating system for rapid free radical polymerization at mild temperatures is, for example, formed from Ag⁺ and acid peroxydiphosphate or persulfate in aqueous media. This redox initiation system is attractive as silver is used widely as an antimicrobial agent. In reacting DOPA with, for example, a core molecule-LDI conjugate to form the prepolymer, unprotected DOPA may be used as the rate of reaction of the catechol side chain of DOPA is much slower than the rate or reaction of the amine group. Silver nitrate and potassium peroxydiphosphate or silver nitrate and ammonium persulfate may, for example, be used as a redox couple to initiate polymerization of the prepolymers hereof Without limitation to any mechanism, cross-linking pathways for the DOPA-containing compounds are illustrated in FIGS. 1B and 1C for a PEG-(LDI-DOPA)₂ precursor. FIG. 1D illustrates a polymer formed using HDI, PEG and DOPA, wherein the DOPA is activated and conjugated with a matrikine.

As described above, the mobility of the bioactive agent relative to the cells incorporated within the polymeric matrices hereof may, for example, be limited by interacting/attaching the biologically active agent with/to at least one of the biodegradable matrix or the cells. In a number of embodiments, bioactive agents hereof and/or other molecules may, for example, be covalently incorporated into the polymer networks of the present invention by first covalently attaching or conjugating the bioactive agent with DOPA and then curing such DOPA-bioactive agent polymers as described above. Two different schemes may, for example, be used to synthesize two different types of DOPA-bioactive agent polymers. In a first synthetic scheme for DOPA and a matrikine such as TNC, a DOPA and TNC only conjugate may synthesized. In this reaction scheme, the amine group in DOPA may be first protected using, for example, trifluoroacetate. The protected DOPA may then reacted with N-hydroxysuccine to synthesize an active DOPA as described in U.S. Pat. No. 8,029,774 and PCT International Patent Application Publication No. WO/2007/005792. The active DOPA may then be reacted with amine groups in the matrikine. The protected amine of the DOPA can be unptrotected (using techniques known in the chemical arts) or remain protected before polymerization. In a second synthetic scheme, a prepolymer is formed using, for example, HDI and a core molecule such as PEG. DOPA can, for example, be reacted with an HDI-PEG-HDI prepolymer to get DOPA-HDI-PEG-HDI-DOPA prepolymer. The DOPA-containing prepolymer can then be reacted with N-hydroxysuccine and a matrikine as described above. As the amine group of DOPA is reacted with the HDI-PEG-HDI prepolymer, there is no need to deprotect the amine group as described above in the first synthetic scheme.

Bioactive agents such as matrikines may also be incorporated into polymer matrices hereof such that their mobility is limited relative to cells incorporated into the polymer matrices via non-covalent interaction between the one or more components of the polymer matrix with the bioactive agent. Protein-protein interactions, hydrogen bonding, minimization of hydrophobicity, trapping etc. of biologically active agents may, for example, be used to limit the mobility of bioactive agents such as matrikines relate to cells incorporated within the biodegradable polymers hereof.

In a number of embodiments, the bioactive matrikines may thus be covalently attached to the biodegradable polymer, or be non-covalently limited in mobility via interaction with or binding to other moieties which are immobilized within (for example, covalently linked to) the biodegradable polymer. In a number of embodiments, TNC and/or other matrikines (and/or fragments thereof including EGF-like domains) are immobilized (or limited in mobility) relative to cells incorporated with the polymer by protein-protein interactions with, for example, collagen, or by binding to or interacting with sugar moieties (in, for example, hyaluronic acid). In these embodiments, the molecule which interacts with the bioactive agent may be covalently incorporated within the biodegradable polymer matrix.

The mobility of bioactive agent such as matrikines relative to the cells incorporated into polymer gels or matrices hereof can also be limited via interaction between the bioactive agent and the cells. For example, adherence between matrikines and cells can be used to limit mobility of matrikines relative to cells within the polymeric gels or matrices hereof. An agent such as collagen may, for example, be used to enhance matrikine adherence as described above. In such an embodiments, the matrikine adheres to and acts upon the cell that is trapped within the polymer matrix. Entrapment can, for example, be accomplished by varying the polymer density to limit pore size so as to entrap cells with matrikines attached/adhered thereto.

In a number of representative studies of TNC and MSCs incorporated into polymeric gels or matrices hereof, MSCs were found to survive within the degradable polymer gels as described above during the study timeframe and followed an expiration curve that mitigates risk of tumor formation while maximizing their curative functions. Wounded mouse models demonstrated that the polymer gels blunted the localized immune response while increasing neoangiogensis, which are two important aspects of wound healing to be modulated in the human condition. In the same wounded mouse models, the dermis and epidermis matured faster than in non-TNC+MSC polymer gel controls, and present more collagen expressive of wound closure tensile strength.

In several studies, a polyethylene glycol-hexamethylene diisocyanate-dihydroxyphenyl-L-alanine (PEG-(HDI-DOPA)₂ or DHP) pre-polymer-based gel as described above was generated by an independent contract laboratory in the manner described in U.S. Pat. No. 8,029,774 and PCT International Patent Application Publication No. WO/2007/005792. A PEG-DOPA/Collagen prepolymer (in which collagen was covalently incorporated within the prepolymer matrix as described in U.S. Pat. No. 8,029,774 and PCT International Patent Application Publication No. WO/2007/005792) was dissolved in aqueous solution and underwent polymerization initiated by the redox initiation system After combining the pre-polymer and redox catalysts, gelation occurs within a 10 to 20 minute window allowing for sufficient time to incorporate the mesenchymal stem cells. In embodiments in which the cells are to be immobilized within the polymer matrix, the cells may be present during the gelation steps. Gelation proceeded to a viscous solid with a tissue-like consistency.

Cells in culture dishes were coated with Collagen I or with Collagen I and TNC. Human bone marrow-derived multipotential stromal or MSC was seeded on the dishes. The DHB polymer and TNC−MSC were mixed to incorporate the TNC-MCS in the biodegradable polymer.

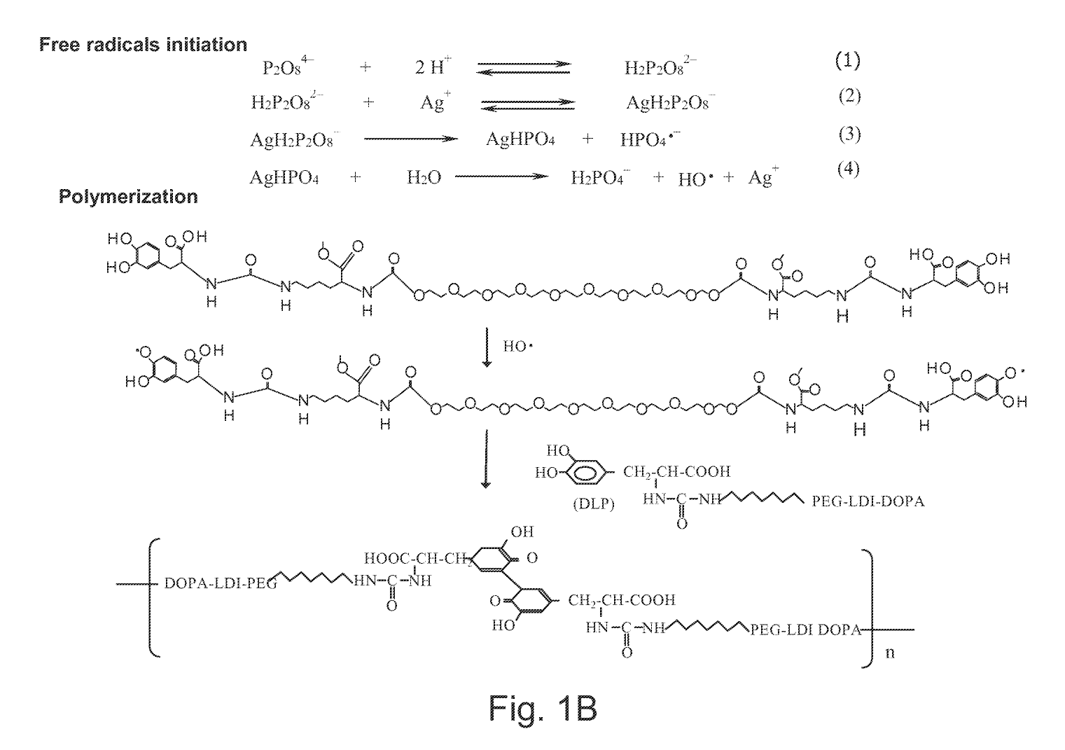

For application of the polymer as a wound-healing matrix, the cured polymer may, for example, remain intact over a number of days or weeks after application. Polymer degradation was tested in vitro by placing 100 mg of polymer into 1 ml of phosphate buffer solution at 37° C. and changing the buffer daily. The rate of degradation was monitored by measuring the amount of intact polymer remaining for up to 10 days. As illustrated in FIG. 2, approximately 60% of the PEG-Polymer+TNC was still intact 10 days after formulation. Phase contrast images of PEG-Polymer+TNC with and without MSC incorporation after 6 days seeding indicated that the gel maintained viable MSCs during the studied time period.

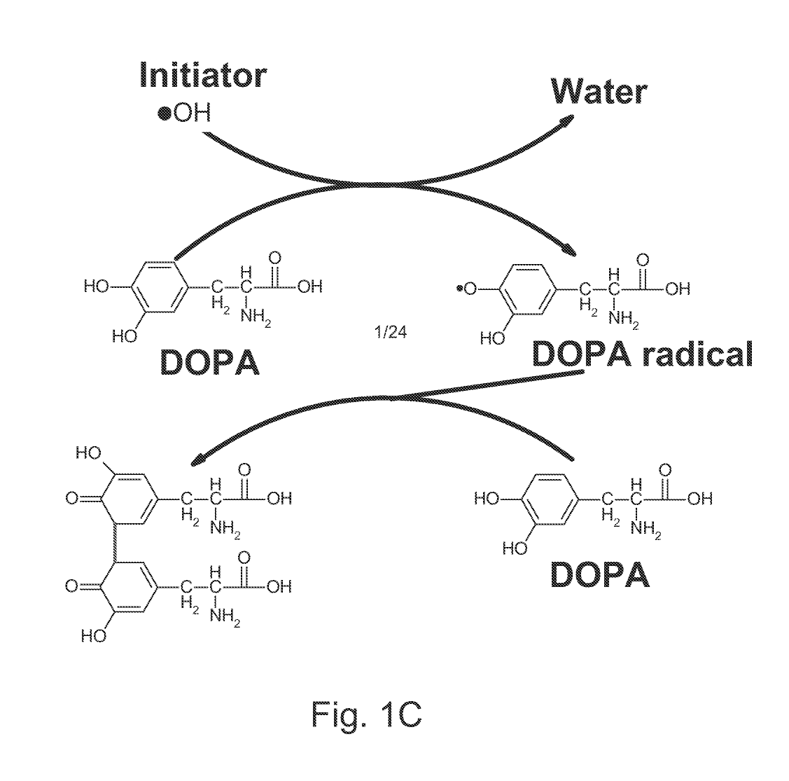

Male and Female FVB (Friend Virus B-Type) and C57BL/6 mice, between 7-9 weeks of age, were used in a number of studies. After shaving the hair on the back of the mice, 8 mm punch biopsies were created. Wounds were either left untreated or treated with commercially available NEW-SKIN® (a liquid bandage or wound protective barrier available from J&J of New Brunswick, N.J.), PEG-Polymer, PEG-Polymer+TNC, or PEG-Polymer+TNC−MSC. As illustrated in FIG. 3, the polymer gels hereof embed within the would bed and remain therein. Thirty minutes after the treatments, the wounds were covered with TEGADERM® (a wound dressing available from 3M of St. Paul, Minn. , and represented schematically by a broken line rectangle in the leftmost photograph of FIG. 3) to maintain uniformity and prevent the mice from removing the treatments. The wounds were examined at 3, 7, 14, 21, 60 and 90 days post-wounding to capture the transitions from inflammatory to regenerative and regenerative to resolving phases of wound healing. Wound bed biopsies surrounded by a margin of non-wounded skin were collected after euthanasia at 3, 7, 14, 21, 60 and 90 days post-wounding.

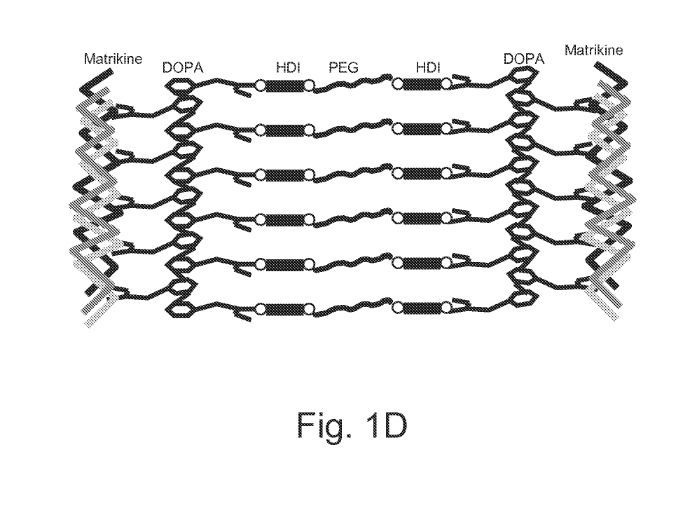

As illustrated in FIGS. 4A and 4B, TNC in the Polymer+TNC/MSC compositions was protective of MSCs in vivo. Flow cytometric quantification of FITC/HLA+cells was used to detect MSC survival in the wound bed. MSCs implanted with PEG-Polymer+TNC hereafter referred to as PEG-Polymer+TNC/MSC were accessed for survival on days 3, 7, and 21. Seven days after treatment with PEG-Polymer+TNC/MSC revealed a 60% survival of MSC in the wound bed. The MSCs thus survive in the gel over 21 days, but decline in cell number. This behavior will prevent both immune response and any issue of tumor formation by the MSC. This was noted in both Wild-Type (WT) mice and mice with a scarring defect (KO).

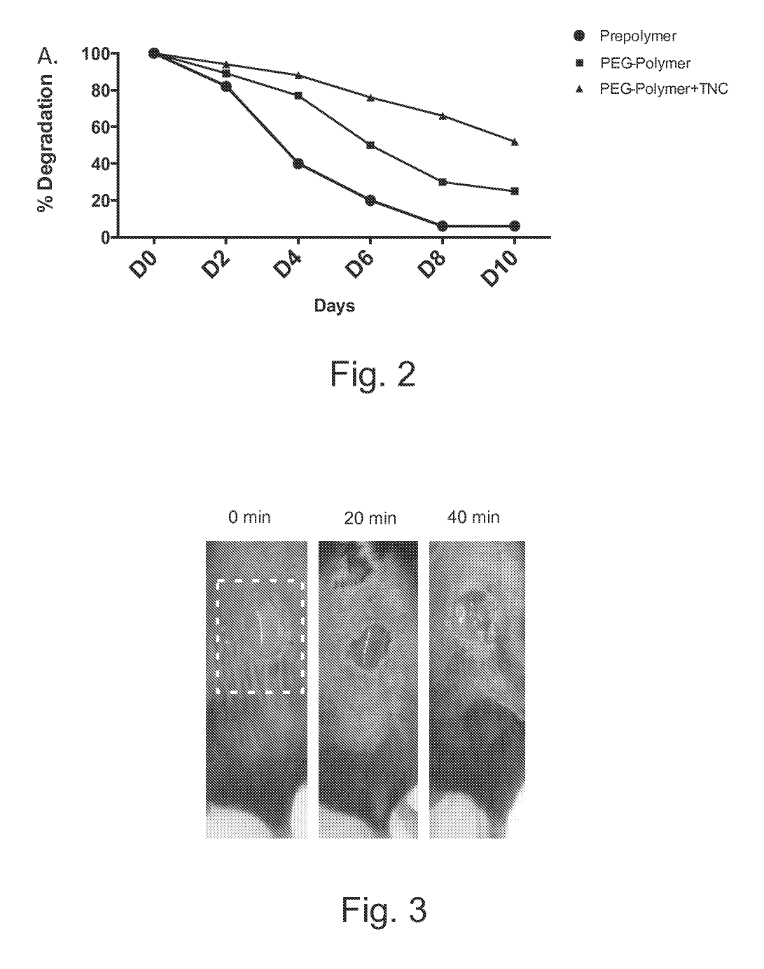

Histopathological examination of mouse tissue was performed for each wound at all time points. Qualitative assessments were made concerning aspects of dermal and epidermal maturation. Samples were scored on a scale of 0 to 4 for epidermal healing (0, no migration; 1, partial migration; 2, complete migration with partial keratinization; 3, complete keratinization; and 4, normal epidermis) and dermal healing (0, no healing; 1, inflammatory infiltrate; 2, granulation tissue present-fibroplasias and angiogenesis; 3, collagen deposition replacing granulation tissue >50%; and 4, complete healing). Measurement of overall wound maturation including epidermal thickness, hyperkeratinization, chronic inflammation, fibroblast cellularity, vascularity, and collagen modeling. As illustrated in FIG. 5, quantification of these wounds revealed that wound maturation in mice that were treated with PEG-Polymer+TNC/MSC show significant improvement in comparison with untreated mice in the first 60 days post wounding.

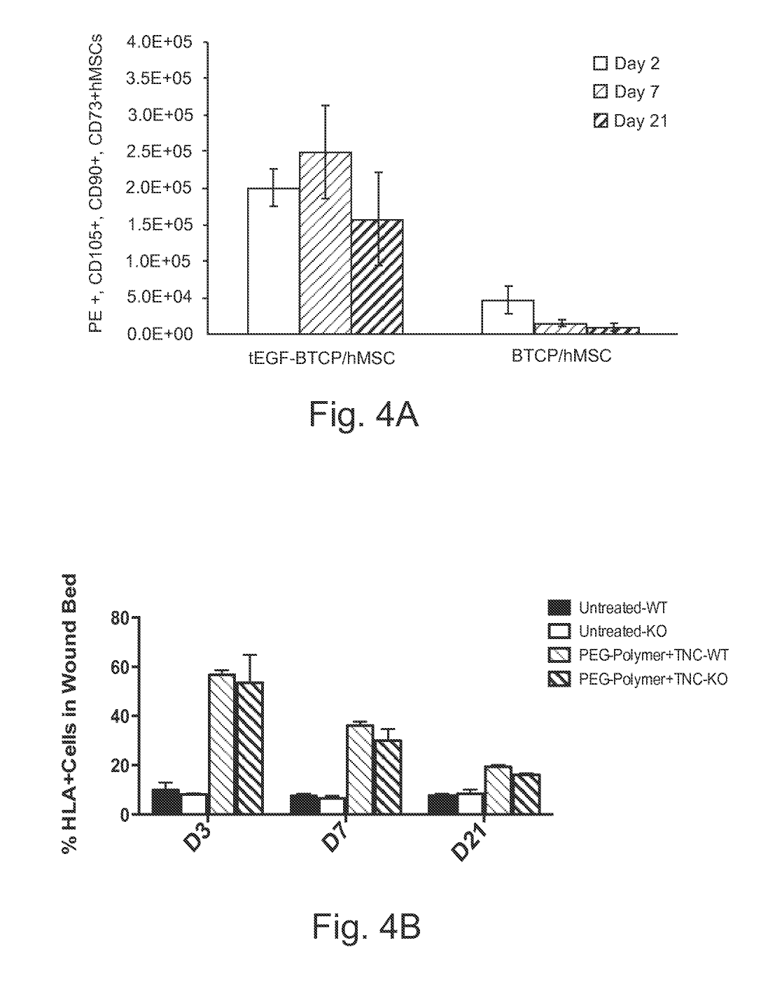

Measurement of dermal maturation determined by examination of collagen remodeling, alignment of the collagen fibrils; fibroblast infiltration in the wounded area was determined by scoring the maturity of the fibroblast from most reactive to normal. As illustrated in FIG. 6A, dermal maturation was more developed and mature in the first 60 days in the PEG-Polymer+TNC/MSC treated wounds. New collagen deposition was quantitatively measured by using Masson's Trichrome microscopic images and MetaMorph analysis of the images of full-thickness wounds, confirming that wounds treated with PEG-Polymer+TNC/MSC resulted in significantly more collagen earlier in the healing process then untreated at days 7 and 21 post-wounding (see FIG. 6B). PEG-Polymer+TNC/MSC treated wounds showed a distinguishably improved dermal maturation patterns at day 60 post-wounding.

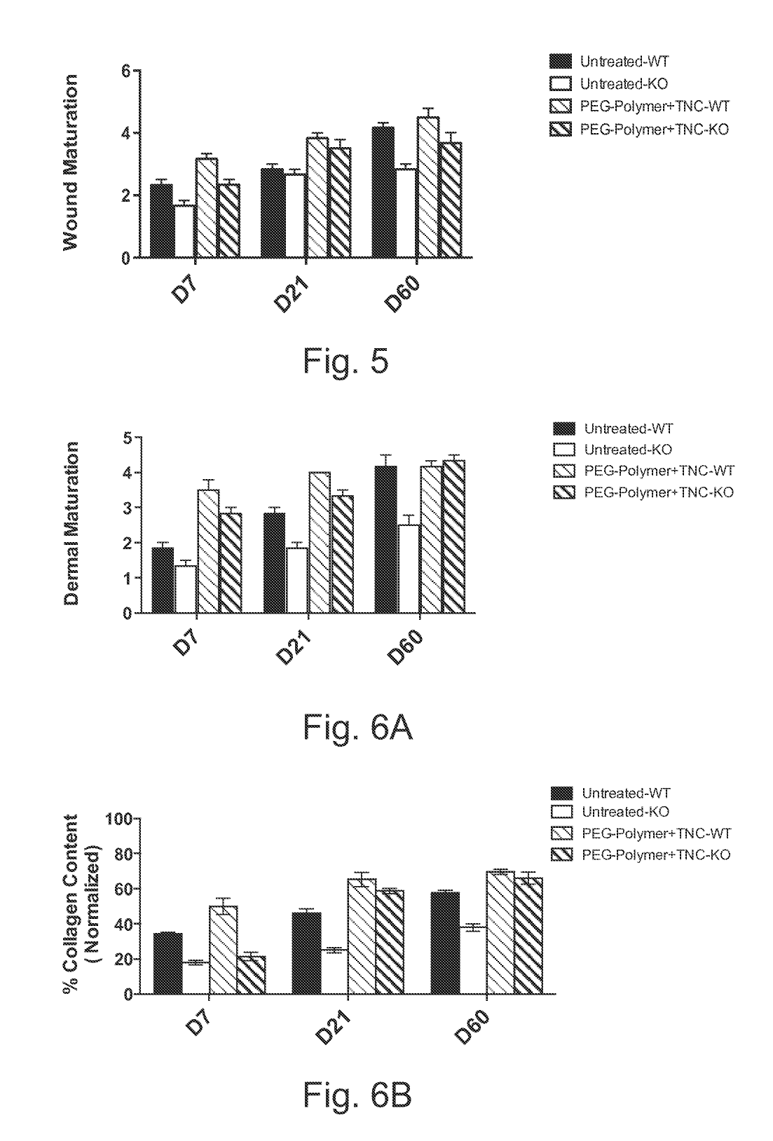

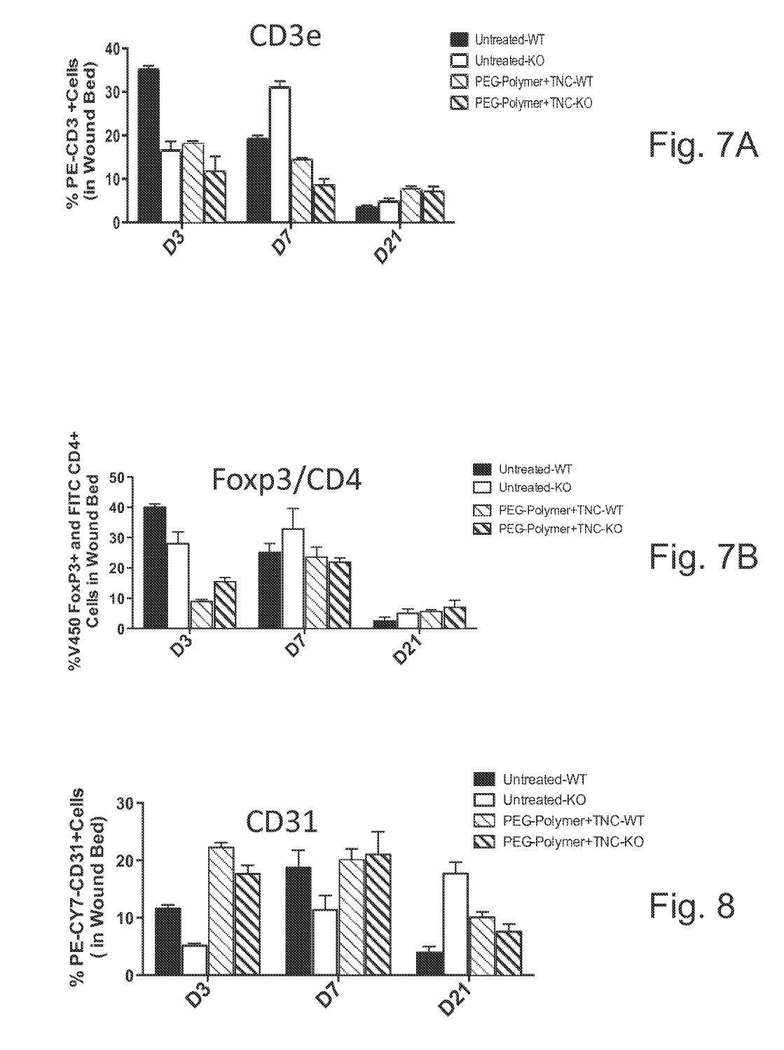

FIGS. 7A and 7B illustrate the results of flow cytometric analysis, which demonstrated that CD3+ and CD4+Foxp3+ expressing cells were limited in the wound bed of wounds treated with the PEG-Polymer+TNC/MSC compared to untreated wound beds. FIGS. 7C and 7D illustrate the results of flow cytometric analysis, which demonstrated that an increase in cells expressing CD31+ and CD44+ in the wound bed at day 3 when wounds were treated with PEG-Polymer+TNC/MSC. The localized immune response (CD3+ and Foxp3+) is blunted, but the angiogenic response (CD31+ and CD44+) is increased within polymer gel and MSC wound samples versus the untreated controls.

### Experimental

Synthesis of polymers. The PEG-(HDI-DOPA)₂ (DHP) pre-polymer-based gel was generated CM-Tec in the manner described in U.S. Pat. No. 8,029,774 and PCT International Patent Application Publication No. WO/2007/005792. In brief, a PEG-DOPA/ColI prepolymer was dissolved in 0.1 m Na₂B₄O₇ aqueous solution (0.3 g prepolymer/ml solution). This underwent polymerization initiated by the redox initiation system AgNO₃/(NH₄)₂S₂O₈ (7 μl/ml gel), yielding 20 μg of Ag/gm polymer gel wet weight. This formulated the PEG-Polymer. Specifics of polymer stability and silver diffusion are discussed in U.S. Pat. No. 8,029,774 and PCT International Patent Application Publication No. WO/2007/005792.

Tenascin C (TNC) and MSC incorporation into PEG-Polymer. Cells in culture dishes were coated for 16 h at 37° C. with 1 μg/cm² Col I, or 1 μg/cm² Col I and 1 μg/cm² TNC diluted in phosphate-buffered saline (PBS). PBS was aspirated, and the coated surfaces were placed under UV light for 10 min before cell seeding. Human bone marrow-derived multipotential stromal (MSC) was seeded on the dish. PEG-Polymer and TNC−MSC was mixed in TEFLON® (a polytetrafluoroethylene polymer available from DuPont of Wilmington, Delaware) dish.

Mouse models for wound healing. Male and Female FVB and C57BL/6 mice, between 7-9 weeks of age, were used for the experiment. All the mice were housed singly and given food and water. They were maintained on a 12-h light and dark cycle at room temperature. The mice were maintained according to the regulations of the Laboratory Animal Welfare Act and amendments and the regulations of the Guide for the Care and Use of Laboratory animals, prepared by the Institute of Laboratory Animal Resources. These studies were deemed approved by the Institutional Animal Care and Use Committee of the Pittsburgh VAMC, where the mice were housed.

After shaving the hair on the back of the mice 8 mm punch biopsies were created. Wounds were either left untreated or treated with commercially available 0.5 ml NEW-SKIN, 0.5 ml final of PEG-Polymer, PEG-Polymer+TNC, or PEG-Polymer+TNC−MSC. Thirty minutes after the treatments, we covered the wounds with TEGADERM in all the five groups to maintain uniformity and prevent the mice from removing the treatments. Wounds were examined the wounds at 3, 7, 14 and 21, 60, 90 days post-wounding. This was done to capture the transitions from inflammatory to regenerative and regenerative to resolving phases of wound healing. Animals were euthanized by CO₂ inhalation and the wounds assessed. For each experiment, in each group at each time point. Each wound was measured and then removed from the animal, with unwounded skin taken from the contralateral dorsum as a control.

Histological analysis. Wound bed biopsies surrounded by a margin of non-wounded skin were collected after euthanasia at 3, 7, 14, 21, 60, and 90 days post-wounding. Wound biopsies were fixed in 10% buffered formalin, processed, and embedded in paraffin blocks using standard protocols. Tissue sections (5 μm) were stained with hematoxylin and eosin, Masson Trichome, P.S red and analyzed for general tissue and cellular morphology. All images were collected at original magnification 10× and 40×.

Epidermal and Dermal Maturation. Histopathological examination of mouse tissue was performed blinded by a trained histopathologist. Qualitative assessments were made concerning aspects of dermal and epidermal maturation as described in Yates et al., “Lack of CXC chemokine receptor 3 (CXCR3) signaling leads to hypertrophic and hypercellular scarring,” American Journal of Pathology, 176: 1743-55 (2010). In brief, the samples were scored on a scale of 0 to 4 for epidermal healing (0, no migration; 1, partial migration; 2, complete migration with partial keratinization; 3, complete keratinization; and 4, normal epidermis) and dermal healing (0, no healing; 1, inflammatory infiltrate; 2, granulation tissue present-fibroplasias and angiogenesis; 3, collagen deposition replacing granulation tissue >50%; and 4, complete healing).

Flow Cytometry. Twelve-millimeter punch biopsies of the wound and 12-mm punch biopsies of unwounded regions were isolated for quantification of MSC engraftment in wounded skin at days 3, 7, 14, and 21. Tissue was minced and incubated in 0.5 mg/mL LIBERASE® TL (an enzyme blend available from Roche Applied Science of Indianapolis, Indiana) for 1 h at 37° C. Tissue was filtered; cells were washed, and labeled for analysis. HLA markers were used to identify positive human MSCs in the wound bed. Appropriate isotype controls, unstained cells, and untreated wounds were used as controls. Flow cytometry was performed on an LSR II Cytometer (a flow cytometer system available from BD Biosciences of Franklin Lakes, N.J.) and subsequently analyzed using FOWJO® digital FACS software (available from Tree Star, Inc. of Ashland, Oreg.).

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A composition comprising a biodegradable matrix, cells within the biodegradable matrix and at least one bioactive agent having limited mobility relative to the cells within the biodegradable matrix, wherein the at least one bioactive agent is bioactive within the biodegradable matrix for regulation of the cells to enhance cell survival.

2. The composition of claim 1 wherein the at least one bioactive agent is a matrikine or a matrikine fragment which is immobilized upon the cells or is immobilized within the biodegradable matrix.

3. The composition of claim 2 wherein the at least one bioactive agent is a matrikine or a matrikine fragment.

4. The composition of claim 3 wherein the cells are cells which can differentiate into at least one other cell type.

5. The composition of claim 3 wherein the cells are multipotent.

6. The composition of claim 4 wherein the cells include at least one of mesenchymal stem cells, tissue stromal cells, tissue epithelial cells or endothelial progenitor cells.

7. The composition of claim 4 further comprising cells or another agent that facilitates or directs differentiation.

8. The composition of claim 4 wherein the matrikine or the matrikine fragment is tethered to the biodegradable matrix.

9. The composition of claim 4 wherein the matrikine or the matrikine fragment is covalently bonded to the biodegradable matrix.

10. The composition of claim 8 wherein the matrikine or the matrikine fragment is non-covalently bonded to the biodegradable matrix.

11. The composition of 4 wherein the matrikine or the matrikine fragment is adhered to the cells.

12. The composition of claim 4 wherein the matrikine or the matrikine fragment is selected from the group of a tenascin, a laminin, a fibronectin, a thrombospondin and an elastin.

13. The composition of claim 4 wherein the matrikine or the matrikine fragment is selected from the group of tenascin-C and laminin beta 1.

14. The composition of claim 4 wherein the matrikine or the matrikine fragment comprises EGF-like repeat regions.

15. The composition of claim 14 wherein the matrikine or the matrikine fragment comprises EGF-like repeat regions of a tenascin or EFG-like repeat regions of a laminin.

16. The composition of claim 14 wherein the matrikine or the matrikine fragment comprises EGF-like repeat regions of tenascin-C or EFG-like repeat regions of a laminin beta 1.

17. The composition of claim 4 wherein the matrikine or the matrikine fragment do not induce differentiation or inhibit differentiation, while the cells are within the biodegradable matrix.

18. The composition of claim 1 wherein the biodegradable matrix comprises a biodegradable polymer matrix.

19. The composition of claim 18 wherein the biodegradable polymer matrix is formed via polymerization a polymerizing reactant that is adapted to undergo a free radical polymerization.

20. The composition of claim 19 wherein the at least one bioactive agent is covalently attached to a polymerizing molecule in forming the polymerizing reactant, the polymerizing molecule retaining the ability to undergo free radical polymerization after attachment of the at least one bioactive agent thereto.

21. The composition of claim 19 wherein at least one interacting agent that is adapted to interact with the at least one bioactive agent is covalently attached to a polymerizing molecule in forming the polymerizing reactant, the polymerizing molecule retaining the ability to undergo free radical polymerization after attachment of the at least one interactive agent thereto.

22. The composition of claim 18 wherein the polymerizing reactant comprises a residue of dihydroxyphenyl-L-alanine (DOPA), a derivative of dihydroxyphenyl-L-alanine, histidine, a derivative of histidine, lysine, a derivative of lysine, tryptophan, a derivative of tryptophan, tyrosine or a derivative of tyorsine.

23. The composition of claim 22 wherein the polymerizing reactant comprises a residue of dihydroxyphenyl-L-alanine (DOPA).

24. The composition of claim 18 wherein the biodegradable polymer matrix comprises a polymer of polyethylene glycol and dihydroxyphenyl-L-alanine (DOPA) or a derivative of dihydroxyphenyl-L-alanine.

25. The composition of claim 18 wherein the biodegradable polymer matrix comprises a polymer of polylactic acid.

26. A method of forming a composition, comprising: incorporating at least one bioactive agent within a biodegradable matrix, incorporating cells within the biodegradable matrix, wherein the at least one bioactive agent can come into contact with the cells and has limited mobility relative to the cells, and wherein the at least one bioactive agent is bioactive within the biodegradable matrix for regulation of the cells to enhance cell survival.

27-51. (canceled)

52. A tissue application system comprising a composition comprising a biodegradable matrix, cells within the biodegradable matrix and at least one bioactive agent having limited mobility relative to the cells within the biodegradable matrix, wherein the at least one bioactive agent is bioactive within the biodegradable matrix for regulation of the cells to enhance cell survival.

53. The tissue application system of claim 52 further comprising a cover adapted to be placed over the composition upon application of the composition to tissue.
